(19) United States
(12) Patent Application Publication
(10) Pub. No.: US 2026/0183468 A1
(43) Pub. Date: Jul. 2, 2026

(54) Dynamic Pressure Response System

(71) Applicant: C. R. Bard, Inc., Franklin Lakes, NJ (US)

(72) Inventors: Jason Jishen Cheng, Avondale Estates, GA (US); David M. Simiele, Roswell, GA (US)

(21) Appl. No.: 19/547,569

(22) Filed: Feb. 23, 2026

Related U.S. Application Data
(63) Continuation of application No. 18/036,335, now Pat. No. 12,558,474.

(51) Int. Cl. A61M 1/00; A61M 25/00; A61M 39/22; G16H 40/63

(52) U.S. Cl. CPC A61M 1/85; A61M 25/0017; A61M 2039/226; A61M 2202/0496; A61M 2205/3331; A61M 2205/50; G16H 40/63

(57) ABSTRACT

Embodiments disclosed herein are directed to a dynamic pressure response system for fully automated clearing of dependent loops from a fluid drainage system. Fluid drainage systems include a flexible drainage tube providing fluid communication with a collection container. Dependent loops can form within the tube leading to pooling of urine and provide an increased risk in CAUTI. Dynamic pressure response systems can automatically detect the presence of dependent loops and provide a low-rate positive air pressure to clear the columnized fluid. Further, the system can automatically detect mixed fluid states when a noise level of pressure signals increases, the system can then provide high-rate positive air pressure to clear mixed fluid state liquid from the tube lumen.

### PRIORITY

This application is a continuation of U.S. patent application Ser. No. 18/036,335, now U.S. Pat. No. 12,558,474, which is a U.S. national stage application of International Application No. PCT/US 2020/061367, filed Nov. 19, 2020, each of which is incorporated by reference in its entirety into this application.

### SUMMARY

Briefly summarized, embodiments disclosed herein are directed to a dynamic pressure response system for fully automated clearing of dependent loops from a fluid drainage system. Fluid drainage systems include a flexible drainage tube providing fluid communication with a collection container. The flexibility of the drainage tube can form sections of positive incline where drainage fluid can accumulate, also termed “dependent loops.” Fluid pooling within these dependent loops can cause various complications. For example, urine pooling can be a source of catheter associated urinary tract infection (“CAUTI”) causing agents such as bacteria, microbes, and the like. Hospital Acquired Infections (“HAI”), such as CAUTI, are detrimental to the patient, and also incur extra costs in treating these additional complications. Embodiments disclosed herein are directed to automatic clearing of these dependent loops while mitigating damage to the collection system and trauma to the patient.

Disclosed herein is a drainage system configured to drain a fluid from a body of a patient, the drainage system including a drainage tube defining a drainage lumen and configured to provide fluid communication between a catheter and a collection container, a connector providing pressurized air to the drainage lumen, the connector disposed proximate the catheter, a sensor disposed within the drainage lumen, and a controller logic configured to, i) detect a state of a drainage fluid disposed within the lumen, ii) modify a pressure level of the pressurized air provided by the connector, and iii) determine if the drainage lumen is clear of a drainage fluid.

In some embodiments, the drainage system further includes one or more solenoid valves communicatively coupled to the controller and configured to control one of a fluid communication between the catheter and the drainage lumen, or a fluid communication between the connector and the drainage lumen. The one or more solenoid valves configured to control fluid communication between the connector and the drainage lumen modifies a pressure level of pressurized air between 0% and 100%. The one or more solenoid valves configured to control fluid communication between the connector and the drainage lumen modifies a pressure level of pressurized air between a first pressurized air source and a second pressurized air source. The sensor is configured to detect one of an air pressure within the drainage lumen, or a state of the drainage fluid within the drainage lumen.

In some embodiments, the state of the drainage fluid includes one of a columnized fluid state, a mixed fluid state, or a no drainage fluid state. The controller logic is configured to detect one of an absolute pressure or a percentage change of pressure within the tube lumen. The percentage change of pressure is calculated by determining a moving average value of the absolute pressure values over time. The controller logic is configured to calculate a noise level to determine the state of the drainage fluid disposed within the lumen. A high noise level indicates a mixed fluid state and a low noise level with a pressure spike indicates a columnized fluid state. The noise level is calculated from an amplitude of the percentage change of pressure. The noise level is calculated by performing a Fast-Fourier-Transform (FFT) on the absolute fluid pressure values and determining the amplitude of the high frequency noise portion of the overall pressure signal. The controller logic reduces the pressure level of the pressurized air when the noise level drops below a threshold value. In some embodiments the catheter is configured to be disposed within a urethra to drain urine from a bladder of the patient.

Also disclosed is a method of draining a fluid from a patient including, detecting a presence of fluid within a lumen of a drainage tube, the drainage tube configured to provide fluid communication between a catheter and a collection container, determining a state of the fluid within the lumen, and providing one of a first pressure level or a second pressure level of pressurized air to the lumen.

In some embodiments, the method further includes closing a valve disposed between the catheter and the collection container before providing one of the first pressure level or the second pressure level of pressurized air to the connector. In some embodiments, providing one of a first pressure level or a second pressure level of pressurized air includes modifying a valve between a first open position to provide the first pressure level and a second open position to provide the second pressure level. In some embodiments, providing one of the first pressure level or the second pressure level of pressurized air includes modifying a pump between a first speed to provide the first pressure level and a second speed to provide the second pressure level. In some embodiments, the method further includes detecting an absence of fluid within the lumen and closing a valve, the valve configured to control providing the pressurized air to the lumen.

In some embodiments, the method further includes detecting pressure information from within the tube lumen and calculating a noise level from the pressure information to determine the state of the fluid within the lumen. In some embodiments calculating a noise level includes calculating one of a noise amplitude of the absolute fluid pressure values, a Fast-Fourier-Transformation of the absolute fluid pressure values to determine an amplitude of a high frequency noise portion of the overall pressure information, or an amplitude of a moving average of absolute fluid pressure values. In some embodiments, the method further includes comparing the noise level with a threshold value and modifying the pressurized air at the connector from the first pressure level to the second pressure level. In some embodiments the state of the fluid includes one of columnized fluid state or a mixed fluid state. In some embodiments, the catheter is configured to drain the fluid from a bladder of the patient and the fluid is urine.

Also disclosed is a drainage system including, a Foley catheter including at least one opening in a distal section, a urine collection container, and a drainage tube assembly fluidly coupling the Foley catheter and the urine collection container, the drainage tube assembly including, a drainage tube including a drainage lumen, a sensor disposed in the drainage lumen, a connector coupling the drainage tube to the Foley catheter, the connector including an inlet for receiving pressurized air, and a controller logic communicating with the drainage tube assembly configured to, i) detect a state of a drainage fluid disposed within the drainage lumen, ii) modify a pressure level of the pressurized air provided by the connector, and iii) determine if the drainage lumen is clear of a drainage fluid.

In some embodiments, the controller logic is configured to modify a valve disposed within the connector between a closed position and one or more open positions to modify a pressure level of the pressurized air. The controller logic is in communication with a pump configured to provide the pressurized air, the controller logic configured to modify the pressure level of the pressurized air by modifying the speed of the pump. The sensor is configured to detect a pressure of a fluid or a presence of a liquid within the drainage lumen. In some embodiments, detecting the state of the drainage fluid include measuring a pressure within the drainage lumen and calculating a noise level.

In some embodiments a high noise level indicates a mixed fluid state drainage fluid and the controller logic provides a high pressure level of pressurized air. The controller logic provides a low pressure level of pressurized air in response to a trigger, the trigger being predetermined time frame or an action. In some embodiments, calculating a noise level includes one of determining a percentage change in pressure values, determining a percentage change in a moving average of pressure values, or performing a Fast Fourier Transform of pressure values to detect an amplitude of a high frequency noise portion.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exemplary catheter and fluid collection system including a dynamic pressure response system, in accordance with embodiments disclosed herein.

FIG. 2A shows an exemplary columnized fluid state within a drainage tube, in accordance with embodiments disclosed herein.

FIG. 2B shows an exemplary mixed fluid state within a drainage tube, in accordance with embodiments disclosed herein.

FIG. 3 shows a pressure chart for a fluid collection system including a dynamic pressure response system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

To assist in the description of embodiments described herein, a longitudinal axis extends substantially parallel to an axial length of the catheter. A lateral axis extends normal to the longitudinal axis, and a transverse axis extends normal to both the longitudinal and lateral axes. Unless defined otherwise, all technical and scientific terms used herein have the same meaning as commonly understood by those of ordinary skill in the art.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

The term “computerized” generally represents that any corresponding operations are conducted by hardware in combination with software and/or firmware. As used herein, the term “fluid” can include a gas, liquid, or combination thereof.

Embodiments disclosed herein are directed to a dynamic pressure response drainage system including control logic configured to enable fully automated clearing of dependent loops from a drainage tube. The dynamic pressure response drainage system can automatically detect the presence of fluid within the drainage tube, determine a fluid state of the fluid within the drainage tube and modify a positive air pressure to clear the fluid from the drainage tube and into the collection container.

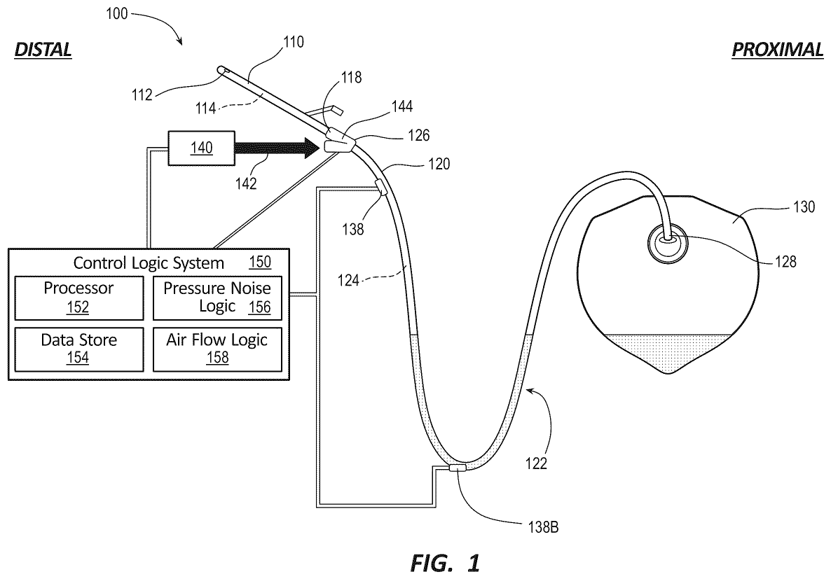

FIG. 1 shows an exemplary dynamic pressure response drainage system (“system”) 100, which generally includes a catheter 110, a drainage tube (“tube”) 120, a collection container (“container”) 130, and a control logic system 150. Exemplary catheters 110 include indwelling catheters, Foley catheters, balloon catheters, peritoneal drainage catheters, or the like, and are configured to be inserted into an orifice within the body of a patient to drain a fluid therefrom. In an embodiment, the catheter 110 can be inserted through the urethra and into a bladder of a patient. The catheter 110 includes an eyelet 112 that provides fluid communication with a lumen 114 of the catheter 110, and is configured to drain a fluid, e.g. urine.

The tube 120 extends from a distal end 126 to a proximal end 128 to define an axial length, and defines a lumen 124. The distal end 126 of the tube 120 can be in fluid communication with a proximal 118 end of the catheter 110. The tube 120 provides fluid communication between the lumen 114 of the catheter 110 and the collection container 130. The tube 120 can be formed of rubber, plastic, polymer, silicone, or similar suitable material. The collection container 130 can include a rigid container, a flexible collection bag, or similar suitable container for receiving a fluid, e.g. urine, drained from the catheter 110.

As shown in FIG. 1, the flexibility of the drainage tube 120 can result in sections of the tube 120 providing a positive incline relative to the direction of fluid flow therethrough. These positive incline portions allow dependent loops 122 to form, which can lead to urine pooling within the tube 120. Urine pooling within the tube 120 can be a source of CAUTI causing agents, e.g. microbes, bacteria, etc. which can be detrimental to the patient. In an embodiment, a source of positive air pressure 140, e.g. a pump, or the like, can introduce a positive air pressure 142 into the tube lumen 124 at a point that is distal to the dependent loop 122. The positive air pressure 142 can urge the fluid through the tube lumen 124 and into the container 130. In an embodiment, the container 130 can include an outlet vent configured to release the positive air pressure within the system 100.

In an embodiment, the system 100 can include a connector piece (“connector”) 144. The connector 144 can include a first inlet configured to couple with an outlet of the catheter 110 and provide fluid communication therebetween, and a second inlet configured to provide fluid communication with the source of positive air pressure 140. The first or the second inlet can be in fluid commination with an outlet of the connector piece 144. The connector outlet can be configured to couple with distal end 126 of the drainage tube 120 and provide fluid communication therebetween. The connector 144 can include one or more valves, e.g. solenoid valves or the like, configured to control a fluid flow between one of the first inlet, second inlet, or outlet of the connector 144. The valve can transition between a closed position and one or more open positions. The one or more open positions can be between 1% open and 100% open. Advantageously, the one or more open positions can provide different rates or pressures of fluid flow therethrough. As such the system 100 can modify the level of positive air pressure 142 entering the tube lumen 124, as described in more detail herein. Further, the system 100 can shut off a fluid flow between the tube lumen 124 and the catheter lumen 114 prior to introducing a positive air pressure 142 to the tube lumen 124 to prevent a distal flow of positive air pressure 142 into the patient. This can prevent trauma to the patient where, for example, the system 100 is activated while the catheter remains in position within the patient.

For example, the connector 144 can include a first valve disposed in the first inlet and configured to control a fluid flow between the catheter lumen 114 and the tube lumen 124. Further, the connector 144 can include a second valve disposed in the second inlet and configured to control a flow of positive air pressure 142 into the tube lumen 124. In an embodiment, the valve of the connector 144 can open to a first open position to provide a first pressure of positive air pressure 142 into the tube lumen 124. The valve can open to a second open position to provide a second pressure of positive air pressure 142, different from the first pressure. In an embodiment the valve of the connector 144 can close to provide no positive air pressure 142 to the tube lumen 124. It will be appreciated that the connector piece 144 can include different numbers or configurations of inlets, outlets, or valves and are contemplated to fall within the scope of the present invention.

In an embodiment, the pump 140 can operate at a first speed to provide a first pressure of positive air pressure 142, or at a second speed to provide a second pressure of positive air pressure 142, different from the first pressure. In an embodiment, the pump 140 can shut down to provide no positive air pressure 142 to the tube lumen 124. In an embodiment, the system 100 can include a first pump providing a first pressure of positive air pressure and a second pump providing a second pressure of positive air pressure different from the first pressure. The connector 144 can be configured to provide one of the first pressure from the first pump, or the second pressure from the second pump to the tube lumen 124. In an embodiment the valve(s) of the connector 144 can close to provide no positive air pressure 142 to the tube lumen 124. It will be appreciated that other numbers and configurations of connector valves 144, pumps 140, or the like, are also contemplated to fall within the scope of the present invention.

The system 100 can further include a dynamic control logic system (“control logic”) 150. The control logic 150 can be communicatively coupled with one of the pump 140, the connector 144, or one or more sensors 138. The control logic 150 can include a processor 152, a data store 154, and one or more logic modules, for example a pressure noise logic module 156 and an air flow logic module 158.

In an embodiment, the pressure noise logic 156 can be communicatively coupled with one or more sensors 138 to detect an absolute pressure level within the tube lumen 124 and calculate a percentage change in pressure (%) to determine a “noise” level. In an embodiment, the air flow logic 158 can be configured to modify a positive air pressure 142 entering the tube lumen 124 between a low-flow and a high-flow. In an embodiment, the air flow logic 158 can be communicatively coupled with one of the pump 140 or the connector 144 to modify a speed of the pump 140 or one or more valves of the connector 144 to modify the positive air pressure 142 entering the system. The control logic 150 can be communicatively coupled with one or more sensors 138 configured to detect a pressure within the tube lumen 124, a state of fluid within the tube lumen 124, combinations thereof, or the like.

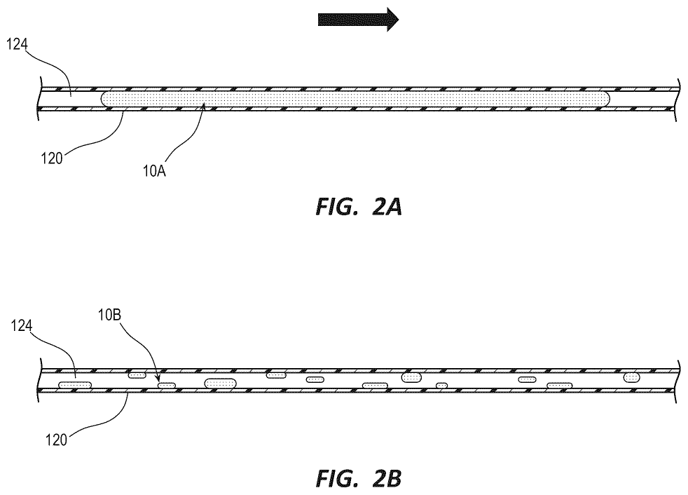

As shown in FIGS. 2A-2B, a fluid 10 disposed within the tube lumen 124 can be categorized into one of three states, a fully columnized fluid state, a mixed fluid state, or no fluid state. As shown in FIG. 2A, a fully columnized fluid state can occur when a liquid within the lumen 124 extends across the entire cross-sectional area of the tube lumen 124. As shown in FIG. 2B, a mixed fluid state can occur when a liquid within the lumen 124 does not extend across the entire cross-sectional area of the tube lumen 124 providing a mixture of gas and liquid within a given portion of tube lumen 124. A no fluid state occurs when little or no liquid, i.e. substantially negligible liquid, remains in the tube lumen 124.

As shown in FIG. 1, for a columnized fluid state, a relatively low static air pressure 142 will push a majority of the fluid within the tube lumen 124, from the dependent loop 122 into the collection container 130, independent of airflow rate. As the amount of liquid within the dependent loop 122 decreases, at a certain point the columnized fluid state will transition to a mixed fluid state where a relatively large positive air pressure 142 flow rate is needed to push the remaining liquid in the mixed flow state, into the collection container 130. As such, the rate of positive air pressure 142 entering the tube lumen 124 needs to change between a low-flow rate for columnized fluid state conditions and a high-flow rate for a mixed flow state conditions. It will be appreciated that high-flow rate and low-flow rate of positive air pressure 142 are exemplary and the system 100 can provide multiple flow rates, e.g. between 0% and 100% of maximum positive air pressure 142, to clear the tube lumen 124 depending on the amount of liquid within the tube, e.g. the number of the dependent loops 122, the column height of the dependent loop 122, the amount or proportion of liquid present in the columnized state or the mixed state, the axial length of tube 120, combinations thereof or the like. Advantageously, the system 100 can provide a positive air pressure 142 between 0% and 100% of maximum positive air pressure sufficient to clear the liquid from the tube lumen 124 without providing excessive positive air pressure that might cause trauma to the patient or damage to the system 100. Further, the system can modify the positive air pressure 142 between 0% and 100% to suit changing conditions within the tube lumen 124.

In an embodiment, the control logic 150 can measure pressure information within the tube lumen 124 and determine one of: the presence or absence of liquid within the tube lumen 124, the presence or absence of a dependent loop 122 within the tube lumen 124, the fluid state conditions, e.g. columnized or mixed state, within the tube lumen 124, a fluid pressure within the tube lumen 124, a fluid pressure spike, a fluid pressure drop, a transition between fluid states within the tube lumen 124, combination thereof, or the like. The control logic 150 can be configured to detect the presence of a dependent loop 122 within the tube lumen 124, modify the operation of one of the pump 140 or a valve system within the connector 144 to apply a positive air pressure 142 distally of the dependent loop 122, determine the presence of a columnized fluid state and apply a low-flow positive air pressure to urge the fluid through the tube lumen 124 and into the collection container 130. The control logic 150 can also determine a transition between the columnized fluid state and the mixed fluid state and modify one of the pump 140 or valve connector 144 to provide a high-flow positive air pressure. The control logic 150 can determine when the drainage tube 120 is sufficiently clear of liquid and either stop the positive air pressure 142 flow at the connector 144, or reduced the positive air pressure 142 flow to a low-flow rate.

It is important to note that a rapid detection of a change in fluid state between the columnized fluid state and the mixed fluid state, and a rapid change between the low-flow and high-flow positive air pressure 142 is important to avoid a large impulse force on the drainage system or the patient catheter 110. Detecting the change and reacting quickly can be important to avoid causing trauma to the patient or damaging the fluid collection system.

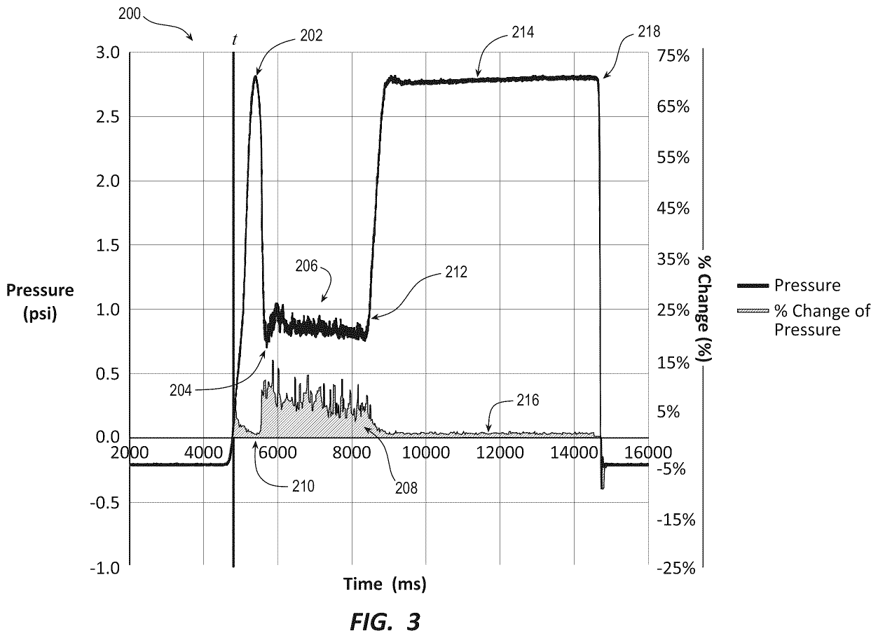

FIG. 3 shows a pressure chart 200 detailing exemplary pressure changes for the system 100 during operation. The pressure chart 200 shows an absolute fluid pressure (“Pressure” in psi) within the tube lumen 124 and a “Percentage change of pressure” (%). In an embodiment, the absolute fluid pressure can be detected by a pressure sensor, e.g. sensor 138, and can be a pressure of a gas, liquid, or combination thereof within the tube lumen 124. In an embodiment, the percentage change of pressure (%) can be calculated by determining a moving average of change in absolute fluid pressure values over time. Advantageously, the span of the moving average (e.g. average of a span of 2 data points, 5 data points, 10 data points, etc.) can be modified to provide different smoothing effects on the data and differentiate the change in noise levels between the different fluid states, as described herein.

In an embodiment, the control logic 150 can determine the fluid state within the tube lumen 124 based on the amount of “noise” in one of the absolute pressure values (psi) or percentage change in pressure (%) values over time. Where a relatively high level of “noise” can indicate a mixed fluid state. As noted, the columnized fluid state requires a relatively low static pressure to urge fluid through the tube lumen since the liquid creates a seal preventing the gas from passing the liquid. Such columnized fluid states can be detected based on a high absolute pressure increase and decrease over a relatively short time span (i.e. a pressure spike) and a low pressure noise (i.e. low cyclical change in pressure). As the liquid in the tube lumen 124 decreases the “seal” created by the columnized liquid can break, causing the columnized fluid to transition into mixed fluid state conditions. In mixed fluid state conditions, liquid droplets can break into smaller droplets or collapse into larger droplets, or columnize and decolumnize rapidly in a cyclical fashion. The positive air pressure 142 within the tube lumen 124 can compress against the cyclical columnizing and decolumnizing of the liquid which causes rapid cycling of pressure levels within the tube lumen 124 leading to a “noisy” pressure signal. In an embodiment, a level of “noise” can be calculated as a relatively high amplitude of percentage change (%) indicating a relatively noisy signal, and a relatively low amplitude of percentage change (%) can indicate a relatively stable signal.

In an embodiment, a value for the percentage change of pressure (%) can be calculated by performing a Fast-Fourier-Transform (FFT) on the absolute fluid pressure values and determining the amplitude of the high frequency noise portion of the overall pressure signal to determine a percentage change value (%). In an embodiment, a value for the level of noise can be calculated by performing a Fast-Fourier-Transform (FFT) on one of the absolute fluid pressure values or the percentage change of pressure (%) values and determining the amplitude of the high frequency noise portion of the signal to determine a “noise” level. As used herein, the “Fast-Fourier-Transform” can break a pressure signal, or percentage change value, down by frequency to isolate the amplitude of the high frequency noise associated with mixed flow states. The control logic 150 can determine the transition from the columnized fluid date to the mixed fluid state based on an increase in a “noise” level, or by comparing a “noise” level relative to a threshold value. The threshold value can be a predetermined value, or a dynamic value determined by the control logic system 150. The control logic 150 can then modify the positive air pressure 142 to a high-flow rate to clear the mixed fluid conditions.

For example, as shown in FIG. 3, a bolus (t) of fluid can enter the tube lumen 124 from the catheter 110 and can collect as a dependent loop 122. The fluid can columnize, blocking a low-flow positive air flow 142 from passing through the tube lumen 124 at the position of the dependent loop 122. In an embodiment, the control logic 150 can apply a constant positive air flow 142 to the tube lumen 124. This causes a pressure spike at 202 as the low-flow positive air pressure 142 builds and forces the columnized fluid through the tube lumen 124. In an embodiment, the control logic 150 can apply a positive air flow 142 to the tube lumen 124 in response to a trigger. The trigger can be a time based trigger or an action based trigger. In an embodiment, the control logic 150 can apply a positive air flow 142 after a given time frame has elapsed. In an embodiment, the control logic 150 can apply a positive air flow 142 in response to an action, e.g. an input from a user, the detection of a liquid within the drainage tube lumen 124, the detection of a dependent loop within the tube lumen 124, or the like.

In an embodiment, the control logic 150 can include a sensor, e.g. sensor 138B, configured to detect the presence or absence of fluid within the tube lumen 124. The control logic 150 can then modify one of the pump 140 or the valve(s) of the connector 144 to apply a low-flow positive air pressure 142 to the tube lumen 124 that compresses against the dependent loop 122 creating the pressure spike 202. Exemplary sensors 138B for detecting the presence of liquid or the presence of a dependent loop 122 can include pressure sensors, humidity sensors, capacitance sensors, or the like. Similarly, the sensor 138B can detect an absence of liquid within the tube lumen 124 to indicate to the controller logic 150 the tube lumen 124 is sufficiently clear of liquid. The positive air pressure 142 can push a fully columnized fluid through the tube lumen 124 and into the collection container 130 creating a rapid increase followed by a rapid decrease in pressure, with low-noise and high pressure amplitude indicating a pressure spike. The control logic 150 can detect the pressure spike to determine or confirm the presence of a columnized fluid state.

In an embodiment, a first valve at a first inlet between the catheter lumen 114 and the tube lumen 124 can be a one-way valve configured to allow a fluid flow to enter the tube lumen 124 from the catheter lumen 114 but prevent any reverse flow, from the tube lumen 124 to the catheter lumen 114. In an embodiment, the first valve can be a solenoid valve, the controller logic 150 can then shut the first valve at the first inlet before the providing a positive air pressure 142 to the tube lumen by way of the second inlet, either by opening the second valve or modifying the speed of the pump 140, or both, as described herein.

At 204, the low-flow positive air flow clears the columnized fluid from the tube lumen 124 and the fluid within the tube lumen 124 transitions from the columnized state to the mixed flow state. As such, the pressure within the lumen 124 drops sharply as the low-flow positive air pressure 142 can pass through the mixed state fluid within the tube lumen 124.

At 206, the remaining fluid within the tube lumen 124 in the mixed fluid conditions can break apart into smaller droplets or collapse into larger droplets, or columnize and decolumnize rapidly, in cyclical fashion, as the low-flow positive air pressure passes through the tube lumen 124. This high frequency, cyclical change fluid conditions can obstruct the tube lumen 124 differently causing high-frequency changes in pressure within the lumen 124 as the positive air pressure 142 passes through the mixed state fluid. These high-frequency changes in pressure create a “noisy” pressure signal, or gives a relatively high amplitude percentage change (%) reading 208, compared with the relative low percentage change (%) under columnized state conditions at 210.

The control logic 150 can detect the relatively high noise pressure signal and determine the transition from the columnized state to mixed flow state within the lumen 124. The control logic 150 can then modify the positive air flow 142 from a low-flow rate to a high flow rate at 212. The high-flow positive air pressure 142 can force the liquid of the mixed state fluid, through the tube lumen 124 and into the collection container 130. The high-flow positive air pressure 142 can provide a consistently high absolute pressure within the tube lumen 214, with relatively low noise, i.e. low amplitude percentage change (%) 216. At 218, the control logic 150 can then determine that the tube lumen 124 is sufficiently clear of liquid and can change the positive air flow from a high-flow rate to a low-flow rate or can stop the positive air flow altogether.

In an embodiment, the control logic 150 can monitor a percentage change (%) and can switch the positive air flow 142 from high-flow to low-flow, or shut off the positive air flow 142 when the noise level drops below a threshold value. In an embodiment, the control logic 150 can operate the high-flow positive air pressure 142 for a predetermined length of time at 214 to determine that the tube lumen 124 is clear. In an embodiment, the control logic 150 receive information from a sensor, e.g. sensor 138B, configured to detect when the tube lumen 124 is sufficiently clear of liquid, the control logic 150 can then reduce or shut off the positive air flow 142 when the tube lumen 124 is sufficiently clear of liquid. The sensor 138B can be a pressure sensor, a humidity sensor, capacitance sensor, or the like.

In an embodiment, the control logic 150 can maintain a continuous positive air pressure 142 through the tube lumen 124. In an embodiment, the control logic 150 can selectively start and a stop the positive air pressure 142 through the tube lumen 124 depending on the presence of fluid within the tube lumen 124 or the presence of dependent loops 122 within the tube lumen. In an embodiment, the control logic 150 can operate autonomously, requiring little or no input from a user, in order to urge fluid through the tube lumen 124 and clear dependent loops 122 therefrom.

Advantageously, the control logic 150 can automatically detect when fluid conditions within the tube lumen 124, transition between columnized and mixed flow conditions and can quickly modify the positive air pressure 142 between low-flow and high-flow. The timing of when the transition of fluid states and changes in flow rates can be important to avoid a large impulse forces on the drainage system and the patient catheter. Advantageously, the control logic 150 can constantly monitor fluid flow states within the tube lumen 124 and can react quickly (e.g. <1 sec.) to changing conditions.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method of draining a fluid from a patient, comprising:
detecting a presence of fluid within a lumen of a drainage tube via a sensor disposed in the lumen, the drainage tube connecting a catheter to a collection container;
providing pressurized air to the drainage tube; and
performing operations by a controller including:
determining a state of the fluid disposed in the lumen by measuring a pressure in the lumen and calculating a noise level;
modifying a pressure level of the pressurized air provided to the drainage tube; and
determining if the drainage lumen is clear of the drainage fluid.

2. The method according to claim 1, wherein a connector is coupled to the drainage tube proximate the catheter, wherein providing pressurized air to the drainage tube comprises sending the pressurized air through the connector.

3. The method according to claim 2, further including one or more solenoid valves communicatively coupled to the controller, further comprising controlling one of a fluid communication between the catheter and the drainage tube, or a fluid communication between the connector and the drainage tube.

4. The method according to claim 3, further comprising modifying the pressure level of the pressurized air between 0% and 100% via the one or more solenoid valves.

5. The method according to claim 1, further comprising providing a first pressure level of pressurized air or a second pressure level of pressurized air to the connector.

6. The method according to claim 5, further comprising modifying the pressure level between the first pressure level and the second pressure level, wherein modifying the pressure level comprises modifying the pressurized air between a first pressurized air source and a second pressurized air source.

7. The method according to claim 5, further comprising modifying the pressure level between the first pressure level and the second pressure level by modifying a pump between a first speed to provide the first pressure level and a second speed to provide the second pressure level.

8. The method according to claim 5, further comprising comparing the noise level with a threshold value and modifying the pressurized air at the connector from the first pressure level to the second pressure level.

9. The method according to claim 1, wherein calculating the noise level comprises calculating one of a noise amplitude of the absolute fluid pressure values, a Fast-Fourier-Transformation of the absolute fluid pressure values to determine an amplitude of a high frequency noise portion of the overall pressure information, or an amplitude of a moving average of absolute fluid pressure values.

10. The method according to claim 1, further comprising closing a valve configured to control the pressurized air to the drainage tube upon detecting an absence of fluid within the lumen of the drainage tube.

11. The method according to claim 1, wherein the state of the fluid includes one of columnized fluid state or a mixed fluid state.

12. The method according to claim 1, wherein the catheter is a Foley catheter including at least one opening in a distal section, wherein the collection container is a urine collection container, the method comprising draining urine from a bladder of the patient.

13. The method according to claim 1, wherein measuring the pressure in the lumen of the drainage tube comprises measuring one of an absolute pressure value or a percentage change of the pressure in the lumen.

14. The method according to claim 13, wherein the percentage change of the pressure in the lumen is calculated by determining a moving average for a plurality of the absolute pressure values over time.
